(19) United States
(12) Patent Application Publication
(10) Pub. No.: US 2024/0399625 A1
(43) Pub. Date: Dec. 5, 2024

(54) Hydrogel Microstructure Arrays, Methods of Making and Uses Thereof

(71) Applicant: McMaster University, Hamilton (CA)

(72) Inventors: Zeinab Hosseinidoust, Hamilton (CA); Lei Tian, Hamilton (CA)

(21) Appl. No.: 18/696,755

(22) PCT Filed: Sep. 29, 2022
(86) PCT No.: PCT/CA2022/051452

§ 371 Date: Mar. 28, 2024

Related U.S. Application Data
(60) Provisional application No. 63/261,803, filed on Sep. 29, 2021.

(51) Int. Cl. B29C 33/42; A61L 26/00; A61L 27/34; A61L 27/36; A61L 27/52; A61L 27/54; B29C 33/38; B29C 33/40; B29C 41/00; B29C 41/02; B29C 41/38; B29C 41/42; B29K 105/00; B29K 105/24; B29L 31/00

(52) U.S. Cl. CPC B29C 33/424; A61L 26/0047; A61L 26/0057; A61L 26/0066; A61L 26/008; A61L 27/34; A61L 27/3637; A61L 27/3687; A61L 27/3691; A61L 27/52; A61L 27/54; B29C 33/3842; B29C 33/40; B29C 41/003; B29C 41/02; B29C 41/38; B29C 41/42; A61L 2300/404; B29K 2089/00; B29K 2105/0035; B29K 2105/0061; B29K 2105/24; B29K 2825/06; B29K 2995/006; B29K 2995/0092; B29L 2031/753

(57) ABSTRACT

This disclosure relates to a hydrogel comprising a crosslinked biomolecule, wherein the hydrogel comprises microscale structures. Also described is a hydrogel comprising an ordered array of semi-spherical microbumps, wherein the hydrogel is bacteria-repellent. Also described is a hierarchically-structured protein hydrogel that inhibits long term attachment of multidrug resistant Staphylococcus aureus up to 100× over a flat hydrogel. Methods of making and uses thereof are also disclosed herein.

### FIELD

The present disclosure relates to hydrogels, and in particular, to hydrogels comprising microscale structures and methods of making and uses thereof.

### BACKGROUND

Substrates with nano-on-micro hierarchical structures have demonstrated superior performance for applications in biosensing, hard tissue implants, and clotting/fouling mitigation, owing partially to the increase in surface area and change in surface energy. For most biomedical applications, where the structured substrate interfaces living tissue, there is increasing evidence that the stiffness of the bio-interface can affect the intracellular molecular processes of cells such as regulation of gene expression and protein secretion, which could in turn impact physiological processes such as tissue regeneration or even response to drugs. Hydrogels have thus become very popular for biomedical applications because of their hydrated, soft, and flexible nature, which can be tuned to closely resemble that of living tissue. Motivated by the performance boost observed in hard materials, recent attempts on applying hierarchical nano-on-micro structures to hydrogels and microgels have expanded the range of applications of these soft, structured substrates to electrode, tissue engineering scaffolds for regenerative medicine, and microparticles for enhanced inhalable or systemic drug delivery.

Published reports on hydrogels decorated with micro/nanostructures have disproportionately focused on synthetic polymers such as polyethylene glycol and polyoligo (ethylene glycol) methyl ether methacrylate. However, for most biomedical engineering applications, the existing body of literature supports the distinct advantages of protein hydrogels, especially albumin-based hydrogels. Aside from natural biocompatibility, biodegradability, and lack of toxicity from unreacted monomers remained in the gel, albumin-based hydrogels can be designed to specifically interact with the surrounding tissue by inserting sequences associated with promoting cellular adhesion or cell growth. Manufacturing protein hydrogels with hierarchical structure, however, is far from trivial. To date, the only method that has been demonstrated to achieve hierarchical nano-on-micro structures on hydrogels has been the shrinking method. In this method, a thermo-sensitive substrate is heat-shrunk, which in-turn creates wrinkle micropatterns on the hydrogel coated on the substrate.¹ The shrinking method is fairly simple and is not dependent on specialized infrastructure. However, this method cannot be applied to protein hydrogels or polymer hydrogels containing heat-sensitive biological agents, e.g., proteins, peptides, VLP's (virus like particles), antibodies, or even antibiotics and most drugs. Furthermore, the wrinkles are anisotropic and disordered, which may reduce consistency/reproducibility especially if the substrate were to be used for sensing applications.

Alternatively, methods demonstrated for micropatterning a hydrogel might, in principle, be applicable to making hierarchical structures on hydrogels. Photolithography combined with in situ photopolymerization is a versatile micropatterning method and allows for design of hydrogel microstructures such as cylinders, stars and shallow dots, but that too is incompatible with proteins and other heat-sensitive molecules. Indirect use of photolithography, namely designing reverse structures on PDMS or using ready-made microfluidic channels (with 3D printing), is in theory compatible with proteins and biological molecules, although no instance of the use of this method has been demonstrated for making hierarchical structures on hydrogels. A simpler, more robust, templating method is the use honeycomb films with ordered micropores as the template. The honeycomb template can be prepared through the breath figure method by exposing a polymer solution to a humid environment,² and can provide an ordered array of over 100,000 pores in a 1-cm² film.³ This method has been demonstrated for micropatterning polymeric hydrogels such as P (NIPAAM),⁴ or PMMA-PEG hydrogels.⁵ As with other methods, the published reports have been entirely focused on synthetic polymers and even there, no hierarchical structuring has been reported.

The background herein is included solely to explain the context of the disclosure. This is not to be taken as an admission that any of the material referred to was published, known, or part of the common general knowledge as of the priority date.

### SUMMARY

Disclosed herein is the development of ordered shape-controllable rounded microscale structures on hydrogels, such as protein hydrogels, using polystyrene microporous templates, and methods of making thereof. Addition of nanogels, for example, made with the same materials as the hydrogels, results in the formation of hierarchical nano-on-micro structures. In embodiments, the structures exhibit increased surface hydrophilicity and bacteria repellency as compared to a flat hydrogel surface composed of the same protein.

In accordance with an aspect of the disclosure, there is provided a hydrogel comprising a crosslinked biomolecule, wherein the hydrogel comprises microscale structures.

In some embodiments, the microscale structures comprise rounded projections.

In some embodiments, the microscale structures comprise spheres, semi-spheres, and/or shallow-arcuate projections.

In some embodiments, the microscale structures form ordered arrays on a surface of the hydrogel.

In some embodiments, the biomolecule is crosslinked with one or more crosslinkers comprising chemical and/or physical crosslinkers.

In some embodiments, the one or more crosslinkers comprises glutaraldehyde, 1-ethyl-3-(3-dimethylaminopropyl) carbodiimide, sulfosuccinimidyl 4-(N-maleimidomethyl) cyclohexane-1-carboxylate or gold nanoparticles.

In some embodiments, the one or more crosslinkers comprises glutaraldehyde.

In some embodiments, the hydrogel further comprises nanoscale features.

In some embodiments, the microscale structures and nanoscale features form hierarchical structures.

In some embodiments, the hydrogel further comprises a heat-sensitive molecule, such as a biological agent.

In some embodiments, the biomolecule is a protein.

In some embodiments, the protein is an albumin, such as bovine serum albumin

In some embodiments, the microscale structures have an aspect ratio of from about 0.05 to about 0.90.

In some embodiments, the hydrogel has a contact angle of from about 10° to about 80°.

In some embodiments, the hydrogel exhibits hydrophilic properties.

In some embodiments, the hydrogel exhibits repellency to bacteria.

In some embodiments, the hydrogel is biodegradable.

In some embodiments, the hydrogel further comprises one or more additives.

In some embodiments, the additives comprise bacteriophages, antibiotics, proteins, peptides, amino acids, carbohydrates, lipids, and/or nucleic acids.

In some embodiments, the bacteriophages self-assemble into bundles.

In some embodiments, the bacteriophages comprise filamentous bacteriophages.

In some embodiments, the bacteriophages comprise Escherichia coli bacteriophages, such as f1, M13, or fd bacteriophages, or combinations thereof.

In some ePCT/CA2022/051452mbodiments, the hydrogel does not kill bacteria.

In some embodiments, the hydrogel inhibits long term attachment of multidrug resistant Staphylococcus aureus up to 100x over a flat hydrogel.

According to another aspect of the disclosure, there is also provided a device or article comprising the hydrogel disclosed herein.

In some embodiments, the hydrogel is on the surface of the device or article.

According to another aspect of the disclosure, there is also provided a biosensor substrate comprising the hydrogel disclosed herein.

In accordance with another aspect of the disclosure, there is also provided a method for making a hydrogel with microscale structures, the method comprising:
mixing a molecule, optionally a biomolecule, with a crosslinker;
depositing at least one layer of the molecule with the crosslinker on an optionally activated surface layer of a mold comprising micropores;
allowing the molecule with a crosslinker to form a hydrogel;
removing the hydrogel from the mold.

In some embodiments, the hydrogel is optionally formed under vacuum.

In some embodiments, activating the surface layer of the mold comprises tuning the hydrophilicity of the surface layer of the mold.

In some embodiments, the micropores are in a honeycomb formation.

In some embodiments, the microscale structures comprise rounded projections.

In some embodiments, the microscale structures comprise spheres, semi-spheres, and/or shallow-arcuate projections of varied sphericity.

In some embodiments, the microscale structures form ordered arrays on a surface of the hydrogel.

In some embodiments, sphericity of the microscale structures is tunable.

In some embodiments, optionally activating a surface layer of the mold and/or optionally forming the hydrogel under vacuum changes the sphericity of the microscale structures.

In some embodiments, the method further comprises depositing a nanogel layer on the surface of the mold before depositing at least one layer of the molecule with the crosslinker on the optionally activated surface layer of the mold.

In some embodiments, the nanogel layer forms nanoscale features.

In some embodiments, the microscale structures and nanoscale features form hierarchical structures.

In some embodiments, roughness of the hierarchical structures is tunable.

In some embodiments, optionally activating the surface layer of the mold and/or optionally forming the hydrogel under vacuum changes the roughness of the hierarchical structures.

In some embodiments, the mold is fabricated by a breath figure method.

In some embodiments, the mold comprises a thermoplastic polymer.

In some embodiments, the mold comprises polystyrene.

In some embodiments, activating the mold comprises introducing oxygen-rich polar functional groups, in, on or over the substrate.

In some embodiments, activating the mold comprises plasma treatment.

In some embodiments, the biomolecule comprises any molecule comprising carbon atoms.

In some embodiments, the biomolecule comprises protein, peptide, amino acid, carbohydrate, lipid, and/or nucleic acid.

In some embodiments, the protein comprises an albumin, optionally at from about 1% to about 10%.

In some embodiments, the crosslinker comprises glutaraldehyde, optionally at from about 0.1% to about 5%.

In some embodiments, forming the hydrogel comprises incubating the biomolecule with a crosslinker on the surface of the mold for a period of time, such as from about 10 minutes to about 1 hour, such as about 30 minutes, at a temperature of from about 4° C. to about 37° C., such as about room temperature.

According to another aspect of the disclosure, there is also provided a hydrogel made by the method disclosed herein.

According to another aspect of the disclosure, there is also provided a hydrogel comprising an ordered array of semi-spherical microbumps, wherein the hydrogel is bacteria-repellent.

In some embodiments, the hydrogel does not kill the bacteria.

According to another aspect of the disclosure, there is also provided a hierarchically-structured protein hydrogel that inhibits long term attachment of multidrug resistant Staphylococcus aureus up to 100x over a flat hydrogel.

According to another aspect of the disclosure, there is also provided a breath figure templating method for creating hydrogels with hierarchically ordered, isotropic, nano-on-micro structures, wherein the method allows for tunable sphericity and roughness of the structures using a single template.

In some embodiments, the sphericity of the structures is controlled by changing plasma coating of the template and using vacuum on the template during a crosslinking reaction.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1 shows preparation of honeycomb template in exemplary embodiments of the disclosure: (A) schematic of breath figure method: (i) apparatus of breath figure method: a sealable chamber containing saturated sodium chloride and a platform above the liquid surface; (ii) water vapor condenses into microdroplets on the polystyrene chloroform solution; (iii) water droplets self-assemble and sink into the polymer solution, followed by the evaporation of water and chloroform; (iv) polystyrene honeycomb film with spherical pores; (B) Scanning Electron Micrograph of polystyrene film surface showing homogeneous circular pores, inset: optical image of the round polystyrene film exhibiting white color, removed from the glass slide; (C) higher magnification view the honeycomb template demonstrating homogeneous pore size; (D) cross-sectional SEM images of polystyrene honeycomb films, showing spherical pores of uniform depth.

FIG. 2 shows preparation of hydrogels with arrays of sphericity-controlled microbumps in exemplary embodiments of the disclosure: (A) schematic diagram of the process of preparing bovine serum albumin (BSA) hydrogel films with microbumps: (i) honeycomb film is plasma-treated to increase hydrophilicity; (ii) BSA solution, mixed with glutaraldehyde, is added to the polystyrene honeycomb film; (iii) BSA solution gels and form a hydrogel cast on honeycomb mold; (iv) once BSA is crosslinked into a hydrogel, the polystyrene template is dissolved off to reveal a free standing hydrogel decorated with microbumps; (B) water contact angle on untreated polystyrene honeycomb film. (C) water contact angle on plasma-coated polystyrene honeycomb film; (D) photo of prepared BSA hydrogel using a honeycomb film as template (scale bar 1 cm); (E) large-scale cross-sectional SEM image of microbumps prepared using plasma-coated substrate in the presence of vacuum (scale bar 100 μm); (F) SEM image of microbumps (scale bar 10 μm) prepared using plasma-coated substrate with vacuum; (G) SEM image of microbumps (scale bar 10 μm) prepared using plasma-coated substrate with no vacuum; (H) SEM image of microbumps (scale bar 10 μm) prepared using none plasma-coated substrate in the presence of vacuum; (I) SEM image of microbumps (scale bar 10 μm) prepared using none plasma-coated substrate with no vacuum; inset in FIGS. 2F, 2G, 2H and 2I shows cross-sectional SEM image of the same hydrogel, respectively (scale bar 5 μm); (J) diameter and aspect ratio of the microbumps with different sphericity.

FIG. 3 shows strain sweep test indicating the storage modulus (G′, filled) and loss modulus (G″, unfilled) for BSA hydrogel and the pre-gelling components (BSA solution and glutaraldehyde solution) in exemplary embodiments of the disclosure.

FIG. 4 shows (A) Size distribution and (B) zeta potential distribution of BSA nanogels in 1 mM KCl solution in exemplary embodiments of the disclosure.

FIG. 5 shows controlling the surface roughness of BSA hydrogel microbumps in exemplary embodiments of the disclosure: (A) schematic diagram of the process of preparing BSA hydrogel films with hierarchical microbumps; (i) BSA nanogel suspension is first added to the polystyrene honeycomb film; (ii) once the nanogels have settled into the micropores, (iii) BSA solution mixed with glutaraldehyde is added to the on top; (iv) when the gel has solidified; (v) the polystyrene template is dissolved off; (B) SEM images of microbumps of a 5% BSA hydrogel film containing 100 μL of nanogels achieved using plasma-coated substrate in the presence of vacuum; (C) SEM images of microbumps of a 5% BSA hydrogel film containing 100 μL of nanogels achieved using plasma-coated substrate and no vacuum; (D) SEM images of microbumps of a 5% BSA hydrogel film containing 100 μL of nanogels achieved using none plasma-coated substrate in the presence of vacuum; (E) SEM images of microbumps of a 5% BSA hydrogel film containing 100 μL of nanogels achieved using none plasma-coated substrate and no vacuum; (F) cross-sectional SEM image of microbumps in FIG. 5E (scale bar for FIGS. 5B, 5C, 5D, 5E and 5F is 10 μm; scale bar for inset of FIG. 5 E is 2 μm).

FIG. 6 shows the preparation of BSA honeycomb hydrogel films in exemplary embodiments of the disclosure: (A) schematic diagram of the process of casting Ecoflex™ secondary template with microbumps; (i) a polystyrene honeycomb film is prepared; (ii) Ecoflex™ solution is cast on polystyrene honeycomb film under vacuum; (iii) after Ecoflex™ solution turns solid, the Ecoflex™ film can be peeled off from the template then the Ecoflex™ film with microbumps can be peeled from the honeycomb film; (B) SEM image of Ecoflex™ film with microbumps (scale bar 50 μm); (C) schematic diagram of the process of preparing BSA honeycomb film with shallow pores; (i, ii) BSA solution mixed with glutaraldehyde is added to the top of Ecoflex™ microbumps, (iii) and the Ecoflex™ template is subsequently peeled off; (D) SEM image of BSA honeycomb film with shallow pores (scale bar 20 μm); (E) schematic diagram of the process of preparing BSA honeycomb film with deep pores; (i) the Ecoflex™ template is first stretched to 1.5 times its original length and then (ii) BSA solution mixed with glutaraldehyde is added on top; (iii) independent BSA film is obtained by simply peeling off from the template; (F) SEM image of BSA honeycomb film with deep pores (scale bar 20 μm); inset of FIGS. 6D and 6F show cross-sectional SEM image of the same hydrogel respectively (scale bar 50 μm).

FIG. 7 shows wetting behaviour of a water droplet on BSA hydrogel films with different surfaces: (A) flat surface; (B) smooth microbumps; (C) nano-on-micro hierarchical bumps; SEM images of S. aureus on BSA hydrogel films with different surfaces after incubating 2 days: (D) flat surface; (E) smooth microbumps; (F) nano-on-micro hierarchical bumps (scale bar for FIGS. 7D, 7E and 7F is 10 μm; scale bar for inset of FIG. 7D is 5 μm); and (G) S. aureus distribution density on BSA hydrogel films after incubating for 2 days, p<0.0001, in exemplary embodiments of the disclosure.

FIG. 8 shows SEM images of BSA honeycomb hydrogel films with different microstructural surface incubated with Staph. aureus for 2 days in LB media in exemplary embodiments of the disclosure: (A) deep pores; (B) shallow pores.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “heat-sensitive molecule” as used herein refers to a molecular species, such as a biological agent, that degrades and/or denatures under high temperature conditions. Examples of heat sensitive molecules include, but are not limited to, proteins, peptides, virus like particles, antibodies, nucleic acids, antibiotics and other small molecule and/or biological drugs.

The term “hierarchical” as used herein refers to a material having both microscale and nanoscale structural features on the surface of the material.

The term “room temperature” as used herein refers to a temperature in the range of about 20° C. and about 25° C.

It will be understood that any component defined herein as being included may be explicitly excluded by way of proviso or negative limitation, such as any specific compounds or method steps, whether implicitly or explicitly defined herein.

### II. Compositions and Methods of the Disclosure

The present disclosure describes the development of a breath figure templating technique to create hydrogels, such as protein hydrogels, with hierarchically ordered, isotropic, nano-on-micro structures. Furthermore, the method described herein allows for tunable sphericity and roughness of the microbump structures using a single template.

In embodiments, the result is an ordered array of semi-spherical microbumps (15.4±2.0 μm diameter, over 180,000 bumps/cm²). By changing interfacial properties, 2-dimensional (flat) surface to 2.5-dimensional (shallow microbumps) surface and finally 3-dimensional (spherical microbumps) surface is achieved.

In embodiments, this is demonstrated with bovine serum albumin (BSA), a water-soluble protein which binds drugs or inorganic substances noncovalently, but the method is applicable to any hydrogel and can advantageously be used with heat-sensitive components. Serum albumin is synthesized by the liver and aids in molecular transport of a variety of metabolites including cholesterols, fatty acids, bilirubin, thyroxine, drugs, and toxins. Hydrogels formed from albumin exhibit numerous desirable properties such as high biocompatibility, cell adherence, autofluorescence, and self-healing. It is demonstrated herein that the hydrogels decorated with the hierarchical microbump arrays are more hydrophilic than smooth microbumps, but exhibit remarkable bacteria repellency, mitigating adhesion of Staphylococcus aureus by 100×, which makes these hydrogels suitable for applications prone to bacterial contamination, such as advanced wound care or surgical sealant products.

Accordingly, provided is a hydrogel comprising a crosslinked biomolecule, wherein the hydrogel comprises microscale structures.

In some embodiments, the microscale structures comprise rounded projections.

In some embodiments, the microscale structures comprise spheres, semi-spheres, and/or shallow-arcuate projections. In some embodiments, the microscale structures comprise spheres, semi-spheres, and/or shallow-arcuate projections of varied sphericity.

In some embodiments, the microscale structures comprise rounded depressions. In some embodiments, the microscale structures comprise spheres, semi-spheres, and/or shallow-arcuate depressions.

In some embodiments, the microscale structures form ordered arrays on a surface of the hydrogel. For example, the microscale structures may be in a honeycomb formation.

In some embodiments, the biomolecule is crosslinked with one or more crosslinkers comprising chemical and/or physical crosslinkers. In some embodiments, the biomolecule is crosslinked with one or more crosslinkers comprising polyelectrolytes, nanoparticles and/or nanocrystals. In some embodiments, the one or more crosslinkers comprises glutaraldehyde, 1-ethyl-3-(3-dimethylaminopropyl) carbodiimide, sulfosuccinimidyl 4-(N-maleimidomethyl) cyclohexane-1-carboxylate or gold nanoparticles. In some embodiments, the one or more crosslinkers comprises glutaraldehyde.

In some embodiments, the hydrogel further comprises nanoscale features. In some embodiments, the nanogel comprises the same material as the hydrogel. In some embodiments, the nanogel comprises one or more additives. In aspects, the additives may be heat-sensitive.

In some embodiments, the microscale structures and nanoscale features form hierarchical structures.

It will be understood that the biomolecule may be any molecule comprising carbon atoms. In some embodiments, the biomolecule comprises a biopolymer or monomer thereof.

In some embodiments, the biomolecule is a protein, peptide, amino acid, carbohydrate, lipid, and/or nucleic acid, typically a protein. In some embodiments, the heat-sensitive molecule comprises proteins, peptides, VLP's (virus like particles), antibodies, antibiotics and/or other drugs or small molecules.

In some embodiments, the microscale structures have an aspect ratio of from about 0.05 to about 0.90, such as from about 0.05, about 0.06, about 0.07, about 0.08, about 0.09, or about 0.1 to about 0.65, about 0.70, about 0.75, about 0.80, about 0.85, or about 0.90.

In some embodiments, the hydrogel has a contact angle of from about 10° to about 80°, such as from about 10°, a, peptide, amino acid, carbohydrate, lipid, and/or nucleic acid bout 15°, about 20°, about 25°, about 30°, or about 35° to about 55°, about 60°, about 65°, about 70°, about 75°, or about 80°.

In some embodiments, the hydrogel exhibits hydrophilic properties.

In some embodiments, the hydrogel exhibits repellency to bacteria. In some embodiments, the surface exhibits repellency to bacteria and biofilm formation. In some embodiments, the bacteria are selected from one or more of gram-negative bacteria or gram-positive bacteria. In some embodiments, the bacteria are selected from one or more of Escherichia coli, Staphylococcus species, Streptococcus species, Helicobacter pylori, Clostridium species and meningococcus. In some embodiments, the bacteria are gram-negative bacteria selected from one or more of Escherichia coli, Salmonella typhimurium, Helicobacter pylori, Pseudomonas aeruginosa, Neisseria meningitidis, Klebsiella aerogenes, Shigella sonnei, Brevundimonas diminuta, Hafnia alvei, Yersinia ruckeri, Actinobacillus actinomycetemcomitans, Achromobacter xylosoxidans, Moraxella osloensis, Acinetobacter lwoffi, and Serratia fonticola. In some embodiments, the bacteria are gram-positive bacteria selected from one or more of Listeria monocytogenes, Bacillus subtilis, Clostridium difficile, Staphylococcus aureus, Enterococcus faecalis, Streptococcus pyogenes, Mycoplasma capricolum, Streptomyces violaceoruber, Corynebacterium diphtheria and Nocardia farcinica. In some embodiments, the bacteria are Staphylococcus aureus. In some embodiments, the bacteria are drug resistant, such as multi-drug resistant Staphylococcus aureus (MRSA).

In some embodiments, the hydrogel is biodegradable.

It will be understood that the hydrogel may be any suitable hydrogel. In some embodiments, the hydrogel may comprise bacteriophages. In certain embodiments, the hydrogel is as described in WO 2021/0003582, which is incorporated herein by reference in its entirety.

Briefly, in embodiments, the hydrogel composition comprises optionally cross-linked bacteriophages. In aspects, self-organized bacteriophages, such as filamentous M13, may be used as building blocks for bottom-up synthesis to develop hierarchically-structured soft matter. Typically, these hierarchically-structured hydrogels of self-organized, crosslinked bacteriophage bundles are comprised of hundreds of phage nanofilaments that impart both long-range and micron-scale order. In typical aspects, the hydrogels can adsorb up to 16× their weight in water, such as at least about 1×, about 2×, about 3×, about 4×, about 5×, about 6×, about 7×, about 8×, about 9×, about 10×, about 11×, about 12×, about 13×, about 14×, about 15×, or about 16× their weight in water. In additional or alternative aspects, the hydrogel compositions described herein exhibit advanced properties at room temperature, such as self-healing under biological conditions, autofluorescence in three channels that decays through biodegradation, offering non-destructive imaging capability, and bioactivity in the crosslinked state towards host bacteria. The latter is a particularly powerful property, allowing the development of hydrogels with tunable bioactivity when combined with phage display and/or recombinant DNA technology. In particular aspects, the hydrogels are comprised of two components, namely filamentous bacteriophages and a crosslinker, such as glutaraldehyde. These two components provide the aforementioned properties without functionality from additional components or responsive crosslinkers. Thus, in aspects, additional polymers may be excluded from the compositions described herein. In other aspects, such polymers may be included in the compositions described herein.

Accordingly, described herein is a hydrogel composition comprising a physically and/or chemically cross-linked plurality of bacteriophages with one or more crosslinkers. In some aspects, the plurality of bacteriophages self-assembles into bundles comprising filamentous bacteriophage. The bacteriophages may be of any type and may infect any bacteria, but in typical aspects, the of bacteriophages comprise filamentous bacteriophages. In some aspects, the bacteriophages comprise Escherichia coli bacteriophages including, but not limited to, f1, M13, or fd bacteriophages, or combinations thereof. Typically, the bacteriophages are M13 bacteriophages.

Typically, the hydrogel comprises sufficient bacteriophage to result in one or more of the characteristics described herein. In some aspects, the hydrogel comprises at least about 10⁸ PFU/mL bacteriophage, such as at least about 10⁹ PFU/mL, at least about 10¹⁰ PFU/mL, at least about 10¹¹ PFU/mL, at least about 10¹² PFU/mL, at least about 10¹³ PFU/mL, at least about 10¹⁴ PFU/mL, at least about 10¹⁵ PFU/mL, or at least about 10¹⁶ PFU/mL, such as from about 10⁸ PFU/mL to about 10¹⁶ PFU/mL of bacteriophage. In some aspects, the hydrogel comprises micro- and/or nano-sized particles. In some aspects, the hydrogel is dried to form an aerogel or xerogel. In some aspects, the hydrogel is dried to form an aerogel by critical-point drying or freeze-drying.

In some aspects, the hydrogel is antibacterial. In some aspects, the plurality of bacteriophages of the hydrogel further comprises one or more molecules for cell targeting and/or infectivity. In some aspects, the one or more molecules comprises proteins and peptides. In some aspects, the proteins are antibodies.

In some aspects, the plurality of bacteriophages of the hydrogel comprises genetically engineered bacteriophage. In some aspects, the plurality of bacteriophages comprises genetically engineered bacteriophage for selective target ligand recognition.

In some aspects, the hydrogel composition further comprises a drug or bioactive agent, wherein the drug or bioactive agent is encapsulated within the hydrogel. For example, one or more antibiotics or other antimicrobial agents may be combined in the hydrogel composition and, optionally, the antibiotic and/or antimicrobial agent may act synergistically with the crosslinked bacteriophages to treat and/or prevent an infection. Other agents may be included in the hydrogel additionally or alternatively, such as hemostatic agents in the case of a wound dressing for example, or a therapeutic agent or angiogenic agent in the case of an implant.

In some aspects, the hydrogel composition further comprises multiple bacteriophage strains which can treat various bacterial infections, especially infections caused by diverse bacteria species. In other aspects, the hydrogel composition comprises multiple bacteriophage strains for treating a single infection.

In certain aspects, the hydrogel repels but does not kill bacteria. In other aspects, the hydrogel repels and kills bacteria.

In some aspects, the hydrogel inhibits long term attachment of multidrug resistant Staphylococcus aureus up to 100× over a flat hydrogel.

Also provided is a device or article comprising the hydrogel disclosed herein. In some embodiments, the hydrogel is on the surface of the device or article. In some embodiments, the device or article is selected from any healthcare and laboratory device, personal protection equipment and medical device. In some embodiments, the device or article is an advanced wound care or surgical sealant product, such as a mesh, a wound dressing, a graft or an implant. In some embodiments, the device is used for cell culture. In some embodiments, the device is a sensor, such as a biosensor.

Also provided is a biosensor substrate comprising the hydrogel disclosed herein.

Provided herein is also a method for making a hydrogel with microscale structures, the method comprising:
mixing a molecule, optionally a biomolecule, with a crosslinker;
depositing at least one layer of the molecule with the crosslinker on an optionally activated surface layer of a mold comprising micropores;
allowing the molecule with a crosslinker to form a hydrogel;
removing the hydrogel from the mold.

In some embodiments, the hydrogel is optionally formed under vacuum.

In some embodiments, activating the surface layer of the mold comprises tuning the hydrophilicity of the surface layer of the mold.

In some embodiments, the micropores are in a honeycomb formation.

In some embodiments, the microscale structures comprise rounded projections.

In some embodiments, the microscale structures comprise spheres, semi-spheres, and/or shallow-arcuate projections.

In some embodiments, the microscale structures comprise spheres, semi-spheres, and/or shallow-arcuate projections of varied sphericity.

In some embodiments, the microscale structures form ordered arrays on a surface of the hydrogel. In some embodiments, the ordered arrays are a pattern transferred from a mold comprising ordered arrays of honeycomb-shaped micropores.

In some embodiments, sphericity of the microscale structures is tunable. In some embodiments, optionally activating a surface layer of the mold and/or optionally forming the hydrogel under vacuum changes the sphericity of the microscale structures. In some embodiments, omitting the step of activating the surface layer reduces the sphericity of the microscale structures. In some embodiments, forming the hydrogel under vacuum increases the sphericity of the microscale structures.

In some embodiments, the method further comprises depositing a nanogel layer on the surface of the mold before depositing at least one layer of the molecule with the crosslinker on the optionally activated surface layer of the mold.

In some embodiments, the nanogel layer forms nanoscale features. In some embodiments, the nanogel comprises the same material as the hydrogel. In some embodiments, the nanogel comprises an optionally crosslinked heat-sensitive molecule.

In some embodiments, the microscale structures and nanoscale features form hierarchical structures. In some embodiments, roughness of the hierarchical structures is tunable. In some embodiments, optionally activating the surface layer of the mold and/or optionally forming the hydrogel under vacuum changes the roughness of the hierarchical structures. In some embodiments, omitting the step of activating the surface layer increases the roughness of the hierarchical structures. In some embodiments, forming the hydrogel under vacuum increases the roughness of the hierarchical structures.

In some embodiments, the mold is fabricated by a breath figure method. In some embodiments, the mold comprises a thermoplastic polymer. In some embodiments, the mold comprises polystyrene.

In some embodiments, the method further comprises coating the mold with a material to fabricate a reverse mold before depositing at least one layer of the molecule with the crosslinker on the optionally activated surface layer of the mold. In some embodiments, depositing at least one layer of the molecule with the crosslinker on the optionally activated surface layer of the mold is performed using the reverse mold to make microscale structures comprising rounded depressions. In some embodiments, the microscale structures comprise spheres, semi-spheres, and/or shallow-arcuate depressions. In some embodiments, the reverse mold comprises an elastomeric polymer. In some embodiments, the reverse mold comprises Ecoflex™

In some embodiments, activating the mold comprises introducing oxygen-rich polar functional groups, in, on or over the substrate. In some embodiments, oxygen-rich polar functional groups comprise hydroxyl, carboxyl and/or carbonyl groups.

In some embodiments, activating the mold comprises plasma treatment. In some embodiments, the plasma treatment comprises gaseous air, O₂, CO₂, allylamine plasma, ammonia plasma, and/or nitrogen plasma.

In some embodiments, the molecule with a crosslinker comprises a polymer or monomer thereof. In some embodiments, the molecule comprises a synthetic polymer or monomer thereof. In some embodiments, the molecule comprises a biopolymer or monomer thereof. In some embodiments, the molecule comprises a heat-sensitive molecule. In some embodiments, the heat-sensitive molecule comprises a biological agent. In some embodiments, the heat-sensitive molecule comprises a biopolymer or monomer thereof. In some embodiments, the heat-sensitive molecule comprises a biomolecule. In some embodiments, the heat-sensitive molecule comprises a protein. In some embodiments, the protein comprises an albumin, optionally at from about 1% to about 10%, such as about 1%, about 2.5%, about 5%, about 7.5% or about 10%.

In some embodiments, the crosslinker comprises glutaraldehyde, optionally at from about 0.1% to about 5%, such as about 0.1%, about 0.5%, about 1%, about 2%, about 3%, about 4%, or about 5%.

In some embodiments, forming the hydrogel comprises incubating the biomolecule with a crosslinker on the surface of the mold for a period of time, such as from about 10 minutes to about 1 hour, such as about 30 minutes, at a temperature of from about 4° C. to about 37° C., such as about room temperature.

Also provided is a hydrogel made by the method disclosed herein.

Also provided is a hydrogel comprising an ordered array of semi-spherical microbumps, wherein the hydrogel is bacteria-repellent.

In aspects, wherein the hydrogel does not kill the bacteria. In other aspects, the hydrogel does kill bacteria.

Also provided herein is a hierarchically-structured protein hydrogel that inhibits long term attachment of multidrug resistant Staphylococcus aureus up to 100× over a flat hydrogel.

Also provided herein is a breath figure templating method for creating hydrogels with hierarchically ordered, isotropic, nano-on-micro structures, wherein the method allows for tunable sphericity and roughness of the structures using a single template.

In aspects, the sphericity of the structures is controlled by changing plasma coating of the template and using vacuum on the template during a crosslinking reaction.

### EXAMPLES

The following non-limiting examples are illustrative of the present disclosure: Methods

Materials: Polystyrene (M_{w}=650 000), chloroform, ethanol and toluene were purchased from MilliporeSigma. Bovine serum albumin (BSA; lyophilized, powder), glutaraldehyde (GA) solution (50%) and LB broth were obtained from fisher scientific. Ecoflex™ was purchased from Sculpture Supply Canada. All chemicals were used as received. Staphylococcus aureus MZ100 was generously provided by Dr. Michael Surette, McMaster University.

Preparing polystyrene honeycomb film: A sealed chamber with a removable lid was set up within saturated NaCl solution and a platform above the liquid level to maintain a stable humid environment. 5 wt % of polystyrene (M_{w}=650 k) in chloroform was dropped on a glass slide placed on the platform and the chamber lid was sealed thereafter. The polystyrene solution was allowed to evaporate and turn solid for 20 mins before being taken out, peeled off the glass slide, and stored for imaging and use in further experiments.

Preparing BSA hydrogel film with microbumps: A final concentration of 5% BSA and 1% GA solution was added on to the surface of a plasma-treated polystyrene honeycomb film and left incubating for 30 mins at room temperature to solidify and form a gel. The polystyrene honeycomb film was removed from the BSA hydrogel by submerging the sample in a glass vial of toluene for 20 mins in a shaking incubator. This step was repeated two more times with new glass vials of toluene. The sample was dried with Kimwipes. The sample was then stored in MilliQ water for future imaging and experimentation. BSA hydrogel with flat surface was obtained using a flat polystyrene film as the template where the procedure of removing flat polystyrene film was same as removing polystyrene honeycomb films.

Control the sphericity of microbumps on BSA hydrogel film: To achieve varied sphericity of the microbumps, the polystyrene honeycomb films underwent a combination of either plasma treatment with CO₂ for 10 minutes to increase hydrophilicity or no plasma treatment, and the addition of 5% BSA and 1% GA under vacuum conditions (e.g. about −0.1 to about −0.08 MPa) or normal atmospheric conditions (e.g. 0 MPa).

Synthesizing and characterizing BSA nanogels: The preparation of BSA nanogels followed a classic desolvation method, described by Weber et al.⁶ Briefly, 150 mg of BSA was dissolved in 2 mL of 10 mM NaCl solution, and then 10 μL of NaOH was added to adjust the pH to 7-9. 8 mL of ethanol was added continuously dropwise using a syringe pump (1 mL/min) while stirring for 500 rpm at room temperature. After all the ethanol is added, 176.25 μL of 8% GA (1.175 μL GA/mg BSA) was added to the solution to induce particle crosslinking. The reaction was performed while stirring at 500 rpm for 24 hrs. Once the crosslinking reaction was completed, the sample was centrifuged at 25,000×g for 10 mins. The supernatant was discarded, and the nanoparticles were dispersed in water for characterization and further experiments. Average particle size was measured using dynamic light scattering (DLS) using a Malvern Zeta Sizer Nano ZSP. Zeta potential of BSA nanogels was measured in 1 mM KCl.

Controlling the roughness of microbumps on BSA hydrogel films: To achieve varied surface roughness of the microbumps, the polystyrene honeycomb films underwent a combination of plasma treatment with CO₂ for 10 mins or no plasma treatment, the addition of the BSA solution in vacuum conditions or normal atmospheric conditions, the addition of BSA nanogels, and the addition of 5% BSA with 1% GA as the chemical crosslinker.

Preparing Ecoflex™ film with microbumps: To prepare Ecoflex™ film with microbumps, a polystyrene honeycomb film was firstly placed inside a well of a twelve-well plate. Ecoflex™ components A and B were mixed together in a 1:1 ratio and dropped on top of the polystyrene honeycomb film. Then the plate was placed in vacuum for 20 mins. After 10 mins, the 12-well plate was taken out. The honeycomb film was pushed to the bottom of the well and left on the bench for 4 hrs to solidify. After solidifying, the Ecoflex™ film was taken out of the 12-well plate. The polystyrene film was peeled off to obtain an Ecoflex™ microbump film and stored at room temperature for future imaging and experimentation.

Preparing BSA honeycomb films: An Ecoflex™ microbump film was either unstretched or stretched using an apparatus containing twine, clamps, and an empty petri dish. BSA solution was placed on the surface of the Ecoflex™ microbump film and placed in a vacuum desiccator for 20 mins or until there were visually no more air pockets in the solution. GA was added to the BSA solution to a final concentration of 1% GA and 5% BSA, and the solution was gently mixed. The sample was put under vacuum for another 30 mins and was then taken out and left on the bench for an additional 1 hr to continue gelation. Once gelled, the BSA hydrogel was peeled and kept in MilliQ water for future imaging and experimentation.

Bacterial adhesion test: Overnight culture of S. aureus was diluted 1000× (10 μL of overnight culture in 10 mL of LB media), added to 3 BSA hydrogel films with or without microbumps and incubated at 37° C., 180 rpm for 48 hrs. Then the hydrogels were taken out and washed for electron microscopy. 8 images were counted for bacteria quantification.

Hydrogel characterization: The morphologies of the honeycomb films and microstructured hydrogels were coated with a 15 nm layer of gold and imaged by a scanning electron microscopy (TESCAN VP, SEM) under 10 kV. The hydrogels were processed critical-point drying before imaging. The water contact angles of hydrogel surfaces were measured by a contact angle instrument (KRUSS, Drop Shape Analysis System DSA 10) with water droplets (5 μL) dispensed by automated syringe.

### Results and Discussion

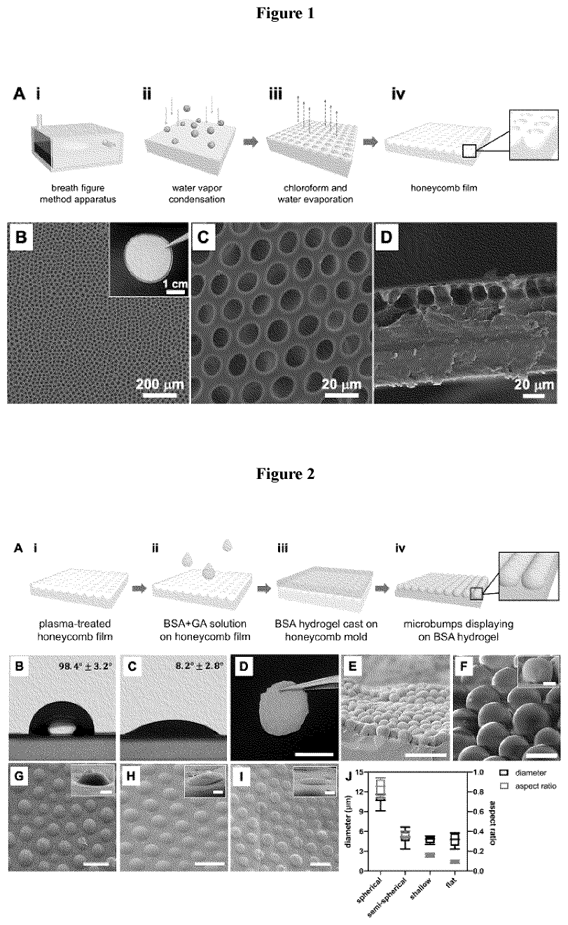

Templates of polystyrene honeycomb films were prepared with the static breath figure method (FIG. 1A). A stable humid environment (relative humidity≈76%) was established inside a sealed chamber (FIG. 1A-i). In the breath figure method, as a result of chloroform evaporation, the surface temperature of a liquid polystyrene film (5 wt % polystyrene, M_{w}=650 000) drops drastically, resulting in the condensation of water vapor into water microdroplets at the liquid-air interface. The microdroplets then sink into the polystyrene solution and self-assemble. In the experiments performed herein, after complete evaporation of water and chloroform, a polystyrene film with a diameter of 2.5 cm was obtained and subsequently peeled off from the glass slide. As shown in FIG. 1B (inset), the polystyrene film had a round shape and white opaque color. Scanning electron micrographs showed the film had micropores with a circular opening, distributed over the film homogeneously at a surface density of 1820±140 pores/mm². The average surface pore size of the film was 10.8±0.9 μm (FIG. 1C). The internal pore shape was open spheres with the average inside diameter of 23.3±2.8 μm (along the direction of cross-section) and the aspect ratio of 0.83±0.02 (FIG. 1D).

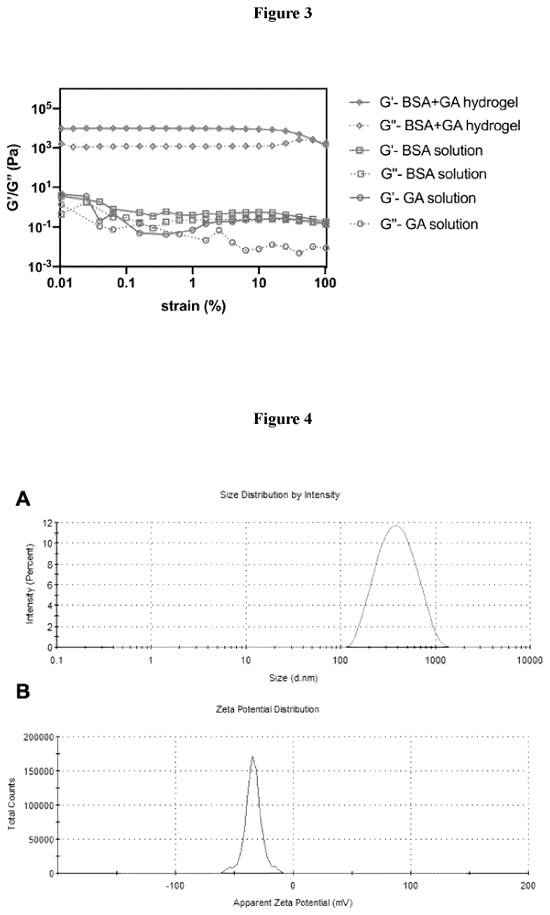

To fabricate BSA hydrogel films with microbumps, a mixture of 5% BSA solution and 1% glutaraldehyde (GA) was cast on a plasma-treated polystyrene honeycomb film, as illustrated in FIG. 2A. The honeycomb film was hydrophobic, as prepared, with a contact angle of 98.4°±3.2° (FIG. 2B). CO₂ plasma is known to place abundant carboxyl groups on the surface of the polystyrene honeycomb film, and so this was used to increase hydrophilicity of the surface and enable the BSA solution to fully occupy the pores. As a result of plasma treatment, the contact angle of water droplets on the polystyrene honeycomb film decreased to 8.2°±2.8° (FIG. 2C). The BSA and GA mixture was added to the plasma-treated polystyrene honeycomb mold under vacuum, which helped the reaction mixture to better fill in the spherical pores of the honeycomb film. After dissolving the polystyrene template in toluene, a stand-alone BSA hydrogel film with microbumps was achieved. The film exhibited a yellow hue (FIG. 2D), typical of BSA crosslinked with GA, with close-packed microbumps (FIG. 2E) and an aspect ratio of 0.85±0.09, close to the value obtained for the pores of the honeycomb film. The SEM image of the insert in FIG. 2F further confirms the sphericity of the microbumps. Characterization of the storage (G′) and loss (G″) moduli of the BSA/GA mixture before and after crosslinking confirmed the gelation of BSA (FIG. 3).

It was further demonstrated that by changing two parameters, namely plasma coating of the template and using vacuum on the template during the crosslinking reaction, the sphericity of microbumps could be controlled. In the absence of vacuum, the shape of microbumps was a shallow arch, even in the presence of the plasma-coated template, with an aspect ratio of 0.36±0.03 (FIG. 2G). When the template was not plasma coated, and hence was hydrophobic, an aspect ratio of 0.17±0.02 was observed when vacuum was used (FIG. 2H) and an aspect ratio of 0.09±0.01 (FIG. 2I) in the absence of vacuum. These microbumps with different sphericity provide an almost 2-dimensional surface (almost flat), 2.5-dimensional surface (shallow microbumps), and ultimately 3-dimensional surface (spherical microbumps), providing different substrate candidates for preventing non-specific adhesion in diverse applications, such as in cell culture and fabricating sensing surfaces with high specific surface area (FIG. 2J).

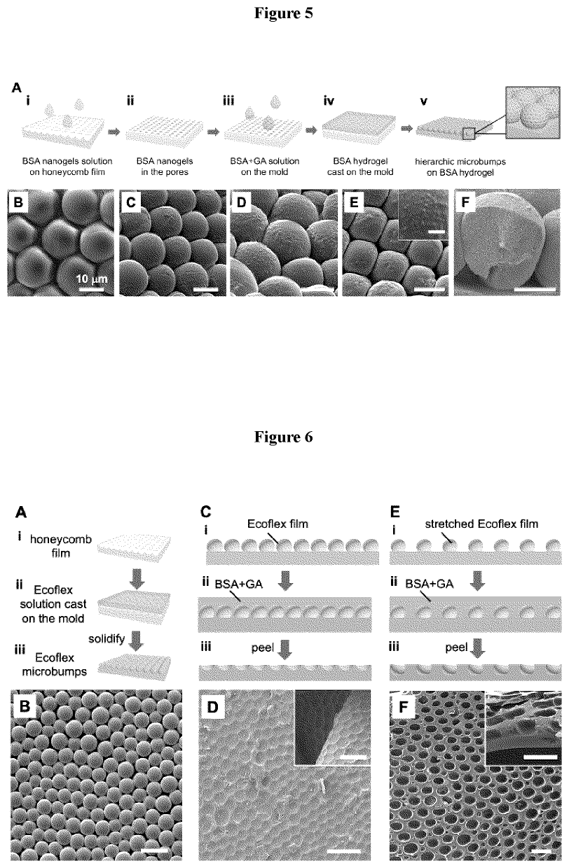

It was demonstrated that BSA microbumps can display nanostructure on their surface after embedding BSA nanogels, which are made of the same components (BSA and glutaraldehyde), inside the bumps during the gelation process. This was achieved by filling BSA nanogels (353.7 nm, ζ potential=−33.8 mV, FIG. 4) into the template pores first, followed by adding BSA and glutaraldehyde mixture to the template (FIG. 5A). Interestingly, no obvious hierarchical structure was observed when a plasma-coated template was used (FIG. 5B), which suggests that the BSA solution completely filled the template pores and encapsulated the nanogels. A nano-on-micro hierarchical structure appeared after omitting vacuum during the crosslinking reaction (FIG. 5C). The surface roughness of the hydrogel microbumps increased further when a none plasma-treated template (hydrophobic template) was used (FIG. 5D), and the highest surface roughness was achieved when both processes were omitted (FIG. 5E). It is noteworthy that the microbumps under all these conditions remained spherical, and the shallow microbumps observed in FIG. 2 were absent. Without being bound by theory, this may be a result of BSA solution filling the gaps between nanogels that had already lined the inside of the micropores as result of capillarity action. FIG. 5F shows a cross section of the microbumps in FIG. 5E (highest roughness); the BSA nanogels appear integrated with the body of the hydrogel, and not as separate layer around the edges of the microbump, yet the microbump surface is visually rough.

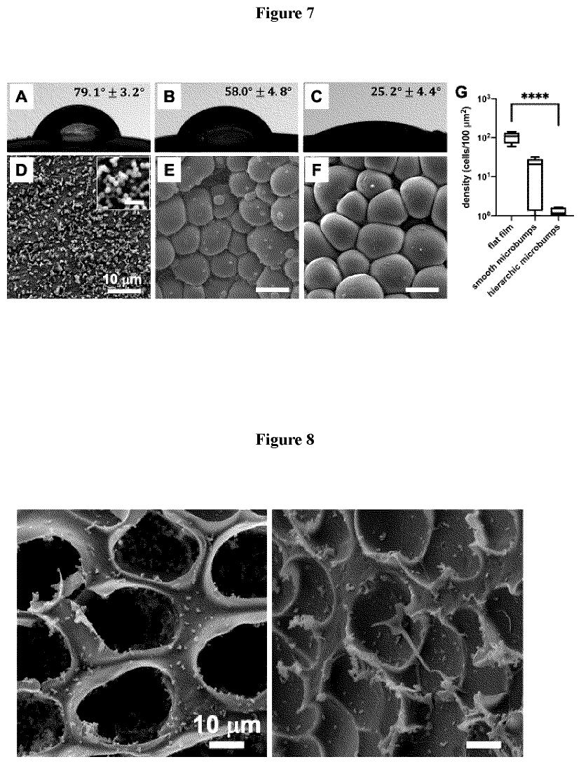

In addition to microbumps, reverse microstructures (honeycomb micropores) on BSA hydrogel films were prepared using Ecoflex microbumps as a secondary template derived from polystyrene honeycomb films (FIG. 6A). The honeycomb hydrogels were prepared as an additional control for investigating the bacterial repellency of the hierarchical microbumps by checking for the effect of reverse microstructures. The material for the secondary template is a silicone elastomer, Ecoflex™, which shows excellent flexibility and stretchability. As shown in FIG. 6B, the Ecoflex™ microbumps are comparable in size to the BSA microbumps, with a size of 27.1±2.3 μm. The BSA hydrogel film was then made by adding BSA and glutaraldehyde solution on the Ecoflex™ template and peeling off the film after gelation (FIG. 6C). The Ecoflex™ template was plasma-coated in advance and vacuum was used during the crosslinking reaction, which resulted in a hydrogel film with very shallow pores (FIG. 6D). This may be due to the gaps between the microbumps being too narrow. As a result, the BSA hydrogel at the bottom of the microbumps were still trapped in the template during the peeling process. The distance between the microbumps can be changed by stretching the Ecoflex™ film. Therefore, the Ecoflex™ template was first stretched in all 4 directions to 2 times its original length. Afterwards, the casting and separation of BSA hydrogel film are same as using unstretched Ecoflex™ template (FIG. 6E). The obtained BSA hydrogel film showed deeper pores with an aspect ratio of 0.57±0.04 (FIG. 6F). To measure hydrophilicity, the contact angle of water droplets on BSA hydrogel films was tested. The flat BSA hydrogel film had a contact angle of 79.1°±3.2° (FIG. 7A), while smooth microbumps had a lower contact angle of 58.0°±4.8° (FIG. 7B). The hierarchical nano-on-micro structure provides a significantly stronger wettability with a contact angle of 25.2°±4.4° (FIG. 7C). Therefore, the microbumps resulted in a more hydrophilic hydrogel surface, with the hierarchical nano-on-micro structure resulting in the most hydrophilic BSA hydrogel surface. It is hypothesized that the increased hydrophilicity of the hierarchical nano-on-micro structure could lead to the formation of a hydration layer, which in turn can exhibit strong bacterial-repellent ability comparing to flat hydrogel surface, based on a similar effect observed in surfaces coated with microgels.⁷ The nanostructured BSA microbumps are expected to reach an highly hydrated state in an aqueous environment, forming a water barrier and preventing the adhesion of the bacteria. To test this hypothesis and to understand the effect of the designed microstructures on bacterial adhesion onto protein hydrogels, S. aureus MZ100 was incubated for 2 days with BSA hydrogel films displaying a flat surface, smooth spherical microbumps, and hierarchical microbumps. The selected S. aureus strain is a multidrug resistant strain known to be a good biofilm former.

As shown in FIG. 7D, the entire flat BSA hydrogel was covered with S. aureus after 2 days of incubation. On the contrary, the bacterial number was visibly less on the smooth spherical microbumps (FIG. 7E). Meanwhile, there were only sporadic occurrences of bacteria adhesion on the hierarchical microbumps (FIG. 7F). The bacterial densities on each surface are presented in FIG. 7G. The distribution density of S. aureus decreased from an average of ˜100 cells/100 μm² for a flat hydrogel surface to ˜1 cells/100 μm² for hierarchical hydrogel microbumps, indicating that bacterial adhesion decreased by 98.9%. The bacterial adhesion on two types of BSA honeycomb films was also tested to rule out the effect of surface heterogeneity on bacteria repellency (FIG. 8). The bacterial density on honeycomb hydrogels is difficult to count because some bacterial cells are sheltered in the pores. However, the bacterial density on the reversed structure is visibly high and on par with flat hydrogel.

In conclusion, the development of hierarchically-structured protein hydrogels that significantly inhibit long term attachment of multidrug resistant Staphylococcus aureus up to 100× over a flat hydrogel is described herein. Reports on bacteria repellent hydrogels are scarce and the few published are based on microstructured (not hierarchically structured) synthetic polymer hydrogels.⁷ Furthermore, none of the previous reports achieved long term repellency and of the magnitude observed herein. By developing a method that enables decorating protein hydrogels with nano-on-micro hierarchical structures, a hydrogel that exhibits bacteria repellency surpassing anything reported in the literature to date was developed. The method for decorating hydrogels with an ordered array of hierarchical structures that is also compatible with protein hydrogels can be extended to polymer hydrogel that contain heat-sensitive molecules, such as antibiotics and most drugs that could in principle be loaded in hydrogels for enhanced delivery. The method developed herein to fabricate uniform hydrogel microstructures, and nano-on-micro structures is much simpler compared to traditional photolithographic or molding methods, which require expensive and complex equipment. This method also allows for the design of hydrogels with different microstructures using the same template. Hydrogels with those microscale patterns, especially with hierarchical structures, provide expanded surface area and hydrophilic surface which are uniquely suited for biosensing as well as antifouling applications.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### CITATIONS

1. K. J. De France, M. Babi, J. Vapaavuori, T. Hoare, J. Moran-Mirabal and E. D. Cranston, ACS Appl. Mater. Interfaces, 2019, 11, 6325-6335.
2. G. Widawski and M. Rawiso, Nature, 1994, 369, 387-389.
3. C. Zhu, L. Tian, J. Liao, X. Zhang and Z. Gu, Adv. Funct. Mater., 2018, 28, 1-8.
4. J. Kyoung and K. C. Basavaraja, Polym. Bull., 2013, 70, 207-220.
5. V. A. Online, J. H. Jang, M. Orba, S. Wang and D. S. Huh, Phys. Chem. Chem. Phys., 2014, 16, 25296-25305.
6. C. Weber, C. Coester, J. Kreuter and K. Langer, Int. J. Pharm., 2000, 194, 91-102.
7. D. Keskin, O. Mergel, H. C. Van Der Mei, H. J. Busscher and P. Van Rijn, Biomacromolecules, 2019, 20, 243-253.

1. A hydrogel comprising a crosslinked biomolecule, wherein the hydrogel comprises microscale structures.

2. The hydrogel of claim 1, wherein the microscale structures comprise rounded projections.

3. The hydrogel of claim 1, wherein the microscale structures comprise spheres, semi-spheres, and/or shallow-arcuate projections.

4. The hydrogel of claim 1, wherein the microscale structures form ordered arrays on a surface of the hydrogel.

5. The hydrogel of claim 1, wherein the biomolecule is crosslinked with one or more crosslinkers comprising chemical and/or physical crosslinkers.

6. The hydrogel of claim 5, wherein the one or more crosslinkers comprises glutaraldehyde, 1-ethyl-3-(3-dimethylaminopropyl) carbodiimide, sulfosuccinimidyl 4-(N-maleimidomethyl) cyclohexane-1-carboxylate or gold nanoparticles.

7. The hydrogel of claim 5, wherein the one or more crosslinkers comprises glutaraldehyde.

8. The hydrogel of claim 1, further comprising nanoscale features.

9. The hydrogel of claim 8, wherein the microscale structures and nanoscale features form hierarchical structures.

10. The hydrogel of claim 1, further comprising a heat-sensitive molecule, such as a biological agent.

11. The hydrogel of claim 1, wherein the biomolecule is a protein.

12. The hydrogel of claim 11, wherein the protein is an albumin.

13. The hydrogel of claim 12, wherein the albumin is bovine serum albumin.

14. The hydrogel of claim 1, wherein the microscale structures have an aspect ratio of from about 0.05 to about 0.90.

15. The hydrogel of claim 1, having a contact angle of from about 10° to about 80°.

16. The hydrogel of claim 1, wherein the hydrogel exhibits hydrophilic properties.

17. The hydrogel of claim 1, wherein the hydrogel exhibits repellency to bacteria.

18. The hydrogel of claim 1, wherein the hydrogel is biodegradable.

19. The hydrogel of claim 1, further comprising additives.

20. The hydrogel of claim 19, wherein the additives comprise bacteriophages, antibiotics, proteins, peptides, amino acids, carbohydrates, lipids, and/or nucleic acids.

21. The hydrogel of claim 19, wherein the bacteriophages self-assemble into bundles.

22. The hydrogel of claim 19, wherein the bacteriophages comprise filamentous bacteriophages.

23. The hydrogel composition of claim 19, wherein the bacteriophages comprise Escherichia coli bacteriophages, such as f1, M13, or fd bacteriophages, or combinations thereof.

24. The hydrogel of claim 1, wherein the hydrogel does not kill bacteria.

25. The hydrogel of claim 1, wherein the hydrogel inhibits long term attachment of multidrug resistant Staphylococcus aureus up to 100× over a flat hydrogel.

26. A device or article comprising the hydrogel of claim 1.

27. The device of claim 26, wherein the hydrogel is on the surface of the device or article.

28. A biosensor substrate comprising the hydrogel of claim 1.

29. A method for making a hydrogel with microscale structures, the method comprising:
a) mixing a molecule, optionally a biomolecule, with a crosslinker;
b) depositing at least one layer of the molecule with the crosslinker on an optionally activated surface layer of a mold comprising micropores;
c) allowing the molecule with a crosslinker to form a hydrogel;
d) removing the hydrogel from the mold.

30. The method of claim 29, wherein the hydrogel is optionally formed under vacuum.

31. The method of claim 29, wherein activating the surface layer of the mold comprises tuning the hydrophilicity of the surface layer of the mold.

32. The method of claim 29, wherein the micropores are in a honeycomb formation.

33. The method of claim 29, wherein the microscale structures comprise rounded projections.

34. The method of claim 29, wherein the microscale structures comprise spheres, semi-spheres, and/or shallow-arcuate projections of varied sphericity.

35. The method of claim 29, wherein the microscale structures form ordered arrays on a surface of the hydrogel.

36. The method of claim 29, wherein sphericity of the microscale structures is tunable.

37. The method of claim 36, whereby optionally activating a surface layer of the mold and/or optionally forming the hydrogel under vacuum changes the sphericity of the microscale structures.

38. The method of claim 29, further comprising depositing a nanogel layer on the surface of the mold before depositing at least one layer of the molecule with the crosslinker on the optionally activated surface layer of the mold.

39. The method of claim 38, wherein the nanogel layer forms nanoscale features.

40. The method of claim 39, wherein the microscale structures and nanoscale features form hierarchical structures.

41. The method of claim 40, wherein roughness of the hierarchical structures is tunable.

42. The method of claim 41, whereby optionally activating the surface layer of the mold and/or optionally performing forming the hydrogel under vacuum changes the roughness of the hierarchical structures.

43. The method of claim 29, wherein the mold is fabricated by a breath figure method.

44. The method of claim 29, wherein the mold comprises a thermoplastic polymer.

45. The method of claim 29, wherein the mold comprises polystyrene.

46. The method of claim 29, wherein activating the mold comprises introducing oxygen-rich polar functional groups, in, on or over the substrate.

47. The method of claim 29, wherein activating the mold comprises plasma treatment.

48. The method of claim 29, wherein the biomolecule comprises any molecule comprising carbon atoms.

49. The method of claim 29, wherein the biomolecule comprises a protein, peptide, amino acid, carbohydrate, lipid, and/or nucleic acid.

50. The method of claim 48, wherein the protein comprises an albumin, optionally at from about 1% to about 10%.

51. The method of claim 29, wherein the crosslinker comprises glutaraldehyde, optionally at from about 0.1% to about 5%.

52. The method of claim 29, wherein forming the hydrogel comprises incubating the biomolecule with a crosslinker on the surface of the mold for a period of time, such as from about 10 minutes to about 1 hour, such as about 30 minutes, at a temperature of from about 4° C. to about 37° C., such as about room temperature.

53. A hydrogel made by the method of claim 29.

54. A hydrogel comprising an ordered array of semi-spherical microbumps, wherein the hydrogel is bacteria-repellent.

55. The hydrogel of claim 53, wherein the hydrogel does not kill the bacteria.

56. A hierarchically-structured protein hydrogel that inhibits long term attachment of multidrug resistant Staphylococcus aureus up to 100× over a flat hydrogel.

57. A breath figure templating method for creating hydrogels with hierarchically ordered, isotropic, nano-on-micro structures, wherein the method allows for tunable sphericity and roughness of the structures using a single template.

58. The method of claim 56, wherein the sphericity of the structures is controlled by changing plasma coating of the template and using vacuum on the template during a crosslinking reaction.
